(19) United States
(12) Patent Application Publication
(10) Pub. No.: US 2020/0330753 A1
(43) Pub. Date: Oct. 22, 2020

(54) ORTHODONTIC TREATMENT

(71) Applicant: Cal-X Stars Business Accelerator, Inc., Playa Vista, CA (US)

(72) Inventors: Howard J. Leonhardt, Corona Del Mar, CA (US); John Joseph Marchetto, Weston, FL (US); Jorge Genovese, Buenos Aires (AR); Alex Richardson, Thousand Oaks, CA (US)

(21) Appl. No.: 16/915,583

(22) Filed: Jun. 29, 2020

Related U.S. Application Data
(63) Continuation-in-part of application No. 15/471,954, filed on Mar. 28, 2017, now Pat. No. 10,695,563.
Application No. 15/471,954 is a continuation-in-part of application No. 29/703,783, filed on Aug. 29, 2019.
(60) Provisional application No. 62/314,240, filed on Mar. 28, 2016.

(51) Int. Cl. A61N 1/32; A61N 1/05; A61C 7/08; A61C 7/12

(52) U.S. Cl. CPC A61N 1/326; A61C 7/12; A61C 7/08; A61N 1/0548

(57) ABSTRACT

Described is a bioelectric stimulating device for reducing orthodontic treatment time (braces or aligners) with post-treatment stability enhancement. The device and associated methods provide a native sustainable optimal upregulated expression and/or release of an increase in the quantity of the right cells and proteins over time and in the right sequence to optimize tooth movement with the braces or aligners by accelerating bone resorption at the leading edge of the tooth during movement. This acceleration phenomenon is responsible for being able to shorten orthodontic treatment time. Following the final alignment of the teeth, the same device can utilize the native response and accelerate the tooth/bone interface stability by targeting specific cells and proteins that are responsible for bone deposition (hardening) in order to shorten the retention phase, while greatly decreasing the chance of relapse (instability).

### CROSS-REFERENCE TO RELATED APPLICATIONS

This application is a continuation-in-part application of co-pending U.S. patent application Ser. No. 15/471,954, filed Mar. 28, 2017, U.S. Pat. No. 10,695,563 (Jun. 30, 2020), which claims the benefit under 35 U.S.C. § 119 of U.S. Provisional Patent Application Ser. No. 62/314,240, filed Mar. 28, 2016; the disclosures of each of which are incorporated herein in their entirety by this reference.

This application is also a continuation-in-part application of co-pending U.S. patent application Ser. No. 29/703,783, filed Aug. 29, 2019, the disclosure of which is incorporated herein in its entirety by this reference.

### FIELD

The application relates generally to the field of dental devices and associated treatments, and more specifically to devices useful for bioelectric stimulation of a subject's tissue to shorten orthodontic treatment time (e.g., treatment with braces or aligners) by accelerating tooth movement and/or enhancing stabilization.

### BACKGROUND

Conventional orthodontic treatment (“braces or aligners”) lasts on average from 18 to 24 months due to the fact that the teeth are housed in bone that must go through the resorption/demineralization (softening) process to allow the teeth to move. The longer the treatment takes, the more side effects are possible, including permanent root length loss and/or gum and bone disease due to improper patient care.

Corticotomy is a widely accepted method for accelerating tooth movement to shorten treatment time, but requires costly bone and gum surgery that can be painful, a short period of acceleration, and has significant associated morbidity.

Following orthodontic treatment, there is a prolonged period of retention while the bone deposition (“hardening”) takes place over the period of up to two years (retention). Orthodontic literature places instability/relapse at 30% or greater. Currently, there is long-term retention using retainers, both fixed and removable, which requires diligence and continued cooperation.

Prior art attempts to shorten orthodontic treatment time have generally proven ineffective, due, e.g., to their inability to significantly increase the rate of tooth movement. Specific protein injection systems to enhance bone resorption in animal studies have experienced a lot of wash out of the therapeutic agent, so continual re-injections are needed, and are thus more painful and are prone to cause infections. Laser therapy systems and vibrational energy systems have been generally ineffective due to a lack of specificity as described in orthodontic literature

### BRIEF SUMMARY

Described is a system (device and method) that provides sustainable protein release and/or expression by a subject with an increase in the quantity of the correct cells and proteins over time and in the right sequence to optimize orthodontic tooth movement by accelerating bone resorption/demineralization at the leading edge of the tooth during movement. This acceleration phenomenon results in shortened orthodontic treatment times (e.g., the amount of time braces or aligners need to be worn by the subject).

Described is a bioelectric device that reduces orthodontic treatment time by, e.g., half (or even more). The device and method provide sustainable optimal release of the cells and protein with an increase in the quantity of the ideal cells and proteins over time and in the right sequence to optimize orthodontic tooth movement by accelerating bone resorption/demineralization at the leading edge of the tooth during orthodontic movement. The bone is then re-mineralized on the trailing edge, and then fully once the teeth are in their corrected orthodontic positions for added stability.

The described system reduces the time necessary to effect a desired tooth movement and reduces the pain associated with tooth movement. It also reduces the tendency of teeth to relapse to their original positions after stopping the orthodontic treatment, and ultimately reduces the time in which unsightly braces need to be worn. The bioelectric stimulator targets the exact native bone resorption pathways that are necessary for tooth movement when an orthodontic force is applied. Specific proteins activate specific cells to cause the cells to initiate bone resorption. This stimulation allows for a greater expression of the specific proteins available that can activate the increased native pluripotent cells. This in turn activates and increases the process of differentiation of pluripotent cells into osteoclasts (bone resorbing cells). With these increases in the targeted bone resorption (softening), the teeth are able to move more rapidly, resulting in an increased rate of tooth movement.

The bioelectric stimulator is also used to enhance bone stability following tooth movement utilizing the bone deposition pathway. In the same manner as described for bone resorption, specific proteins stimulate specific cells to differentiate into osteoblasts (bone deposition cells) and thereby increase the quantity and quality of bone surrounding the teeth after orthodontic tooth movement. This can be done rapidly by expressing the right proteins and cells at the right time to cut the stability time by up to one half.

Also described is a dental or orthodontic mouthpiece having a first portion and a second portion that fold upon one another via a flexible hinge or hinges. When folded, the mouthpiece is sized to fit comfortably within a subject's mouth. Preferably, the mouthpiece contains (and protects from the local environment) circuitry (e.g., a flex circuit) that extends from an electrical interconnection to a contact point or contact points placed to interact with the subject's gums and deliver a bioelectric signal thereto. When an electrical signal (a “bioelectric signal”) is sent through the circuitry, it thus is applied to the subject's gum and bone.

The device can preferably be used at home, for example, with an orthodontist's or dentist's instructions.

Also described is a bioelectric stimulator programmed to activate upregulated expression and/or release (in a subject) of RANKL (for faster treatment), OPG (for better stabilization and retention time), SDF-1 (for modulation of inflammation), VEGF (for modulation of inflammation), and eNOS (as needed).

Bone resorption/deposition is a balance between the amounts of RANKL versus OPG present. When RANKL is signaled for, there is still OPG present, which counteracts some of the RANKL so it is preferred to have significant over expression of RANKL and then conversely for OPG.

Pulsed electromagnetic fields to stimulate OPG and RANKL values are generally too low to make any type of a significant difference. Kanzaki et al. (2004); Kanzaki et al. (2002).

A preferred such system includes:

A bioelectric stimulator that controls/stimulates upregulated expression and/or release/production of, e.g., RANKL, TNF-α, OPG, SDF-1, HGF, IGF-1, VEGF, and eNOS as disclosed in, e.g., U.S. Patent Publication No. 2018/0064935 to Leonhardt et al. (Mar. 8, 2018), the contents of which are incorporated herein by this reference.

The prior art systems fail to produce the correct proteins to attract and produce the right cells in the proper sequence to facilitate consistently increased tooth movement. Existing devices fail to consistently increase the necessary cells and proteins in sequence in order to accelerate the resorption/demineralization (softening) process in bone. Therefore these devices have a limited effect on increasing the rate of tooth movement. For instance, the prior art (e.g., Jansen et al.) did not identify the optimal signals for RANKL and OPG. Their change values were under 30%. There was no control of protein expression. They did not use direct electrical conduction contact with gums to ensure greater signal purity delivery and superior results. There was too much drift in their signal, which in turn can cause bone formation rather than the desired bone resorption.

In the system hereof, the OPG signal directly stimulates osteoprogenitors towards osteogenic differentiation. The RANKL signal in the system hereof also decreases MT1-MMP expression.

Relating to the bioelectric stimulation-controlled upregulated expression and/or release of receptor activator of NFk-B ligand (“RANKL” or “TNFSF11”) among other proteins, including stem cell homing factor SDF-1, designed to accelerate tooth movement and cut in half the time required for orthodontic treatment with braces and clear aligners.

The system addresses the desire to reduce the time it takes to treat orthodontic patients, which would be a boon to them. This approach speeds up the normal process of bone demineralization in order to accelerate tooth movement. Prior art laser light and vibration devices have generally fallen short in providing a reliable pathway to the underlying mechanism of action for tooth movement. Also, experimental repeat RANKL needle injection methods would be painful for patients and needed too frequently. The described system provides clear cut, direct control for the release of essential cells and proteins needed for accelerating tooth movement, and with less pain. Additionally, it can be used in the areas of oral surgery and periodontal surgery for bone grafts to enhance the healing phase of the procedure. Additionally, it can be utilized to enhance the speed for integration of dental implants in bone.

Also, the device is applicable for use in craniofacial surgery where bone grafts are used to repair facial anomalies. Oral surgery can be benefitted by the use of this device for repairing bones in orthognathic surgery, jaw fracture, bone plate insertion, various grafts, and implants. All these areas can benefit from the use of the device because it reduces the amount of discomfort from any of the procedures as the stem cell recruitment and increased vascularity lessens the subject's pain.

### BRIEF DESCRIPTION OF THE DRAWING

FIG. 1 depicts a bioelectric stimulator electrically associated with a mouthpiece as described herein.

FIG. 2 is a perspective view of a dental mouthpiece as described herein.

FIG. 3 is a top view of the dental mouthpiece shown in FIG. 2.

FIG. 4 is a side view of a dental mouthpiece showing leads for delivering the bioelectric signal(s) from the bioelectric stimulator to contact pins for application of the bioelectric signal(s) to the subject's gums.

FIG. 5 is a right side view of the dental mouthpiece shown in FIG. 2.

FIG. 6 depicts a close up view of an embodiment where the contact pin is placed within a connector housing and speared by a lead in a dental mouthpiece as described herein.

FIG. 7 shows the dental mouthpiece of FIG. 4 folded back upon itself, immediately before insertion into the subject's mouth.

### DETAILED DESCRIPTION

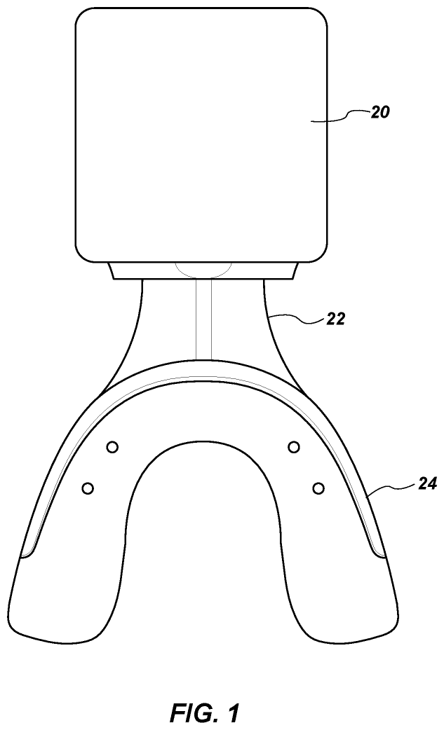

As depicted in FIG. 1, a system useful in an orthodontic procedure comprises a bioelectric stimulator 20 programmed to produce sequential electrical signals in electrical association with (e.g., via stem portion 22) a mouthpiece 24 comprised of a polymer and constructed to fit about and/or over the subject's teeth, braces or clear aligners and in proximity of the subject's gums.

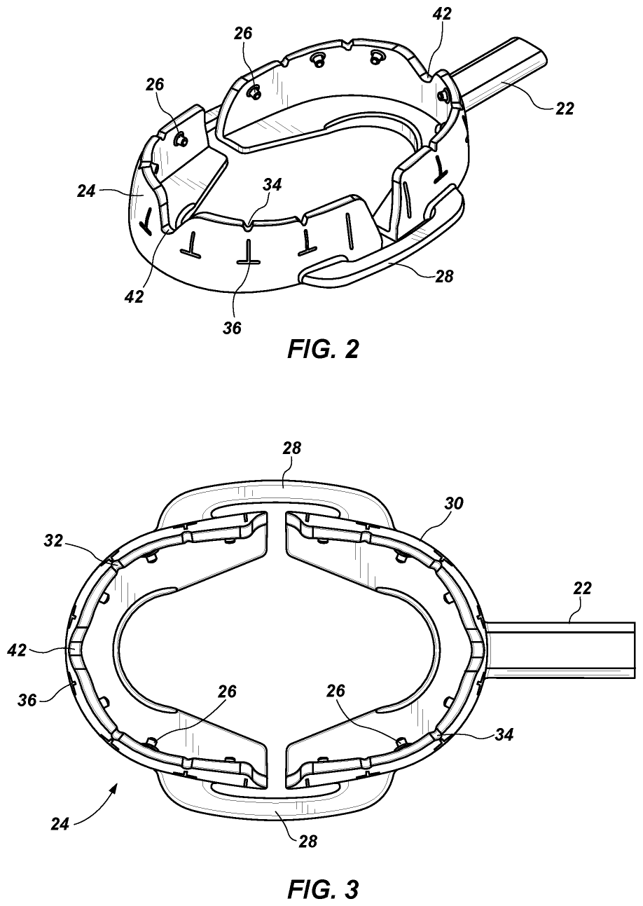

A bioelectric signal generator is used to generate the specific signals typically transmitted via contact pins/points 26 on the mouthpiece 24 (FIG. 2) that cause the specific cells and proteins to be released from cells associated with the gums and bone. The bioelectric stimulator is programmed with selected signals in a designed sequence to facilitate bone resorption/demineralization (softening). In the depicted embodiment, the bioelectric stimulator sends preprogrammed bioelectric signals via the mouthpiece 24 during an orthodontic procedure to the subject's gum and bone tissue.

The bioelectric stimulator can be a micro voltage signal generator produced utilizing the same techniques to produce a standard heart pacemaker well known to a person of ordinary skill in the art. An exemplary microvoltage generator is available (for experimental purpose from Cal-X Stars Business Accelerator, Inc. DBA Leonhardt's Launchpads or Leonhardt Vineyards LLC DBA Leonhardt Ventures of Salt Lake City, Utah, US). The primary difference is the special electrical stimulation signals needed to control (which signals are described later herein). The construction of the electric signal generators, are known in the art and can be obtained from OEM suppliers as well as their accompanying chargers and programmers. The electric signal generators are programmed to produce specific signals to lead to specific protein expressions at precisely the right time for the procedure.

The bioelectric stimulator for use herein can be about the size of two quarters and is programmable. Bioelectric stimulators are commercially available (e.g., from Mettler.)

In certain embodiments, the bioelectric stimulator is programmed to produce either a single electrical signal or, e.g., a sequential train of electrical signals comprising any permutation of the signals described herein to be applied at various patient tolerable amplitudes and for any duration.

In severe cases, a micro pump (not shown, but see the incorporated U.S. Patent Publication No. 2018/0064935 to Leonhardt et al. (Mar. 8, 2018)) may further be utilized to provide a higher volume of therapeutic agents more rapidly.

In certain embodiments (not shown), the bioelectric stimulator is very small and may be incorporated directly into the mouthpiece to avoid the stem portion and connection with a separate bioelectric stimulator.

Further, a bioelectric stimulator may be in contact with, e.g., a smartphone (not shown) via Bluetooth® (Bluetooth SIG, Inc.) to, for example, track wear time and use and to share information with the treating orthodontist.

The mouthpiece 24 shown in FIG. 2 is typically made of a soft, stretchable biocompatible polymer that contains, for example, leads or wiring (see, e.g., FIG. 4) for conducting a bioelectric signal or bioelectric signals from the bioelectric stimulator to contact pins 26 adjacent the subject's gums. The mouthpiece 24 is sized and shaped to fit comfortably when folded upon itself about the subject's teeth. The mouthpiece preferably fits over (or otherwise accommodates) aligners, braces, and/or wires, and covers both arches simultaneously. In the depicted embodiment (see, e.g., FIGS. 2 and 5), there is a positive curvature of the flanges to coincide with the shape of the alveolus of the maxilla and mandible.

The contact pin(s) typically comprise precious or semi-precious contact material(s), and include “Omni Ball” spring loaded contacts, “pogo pins,” “spring probes,” hyberboloid contacts Hypertac, SuperButton, SuperSpring contacts and even a properly shaped “stud” or wire (e.g., copper or beryllium) lead and FUZZ BUTTONS® contact pins. Various other means of accomplishing electrical conductivity in the mouthpiece are known. For example, electrically conductive adhesive tape is available from 3M of Minnesota. Silicone-based Electrically Conductive Adhesive (ECA) has been developed for the Metal Wrap-Through module technology. Conductive polymers are known in the art, and could be, e.g., linear-backbone “polymer blacks” (polyacetylene, polypyrrole, and polyaniline) and their copolymers. See, also, Kaur et al. “Electrically conductive polymers and composites for biomedical applications,” RSC Adv., 2015, 5, 37553-37567 DOI: 10.1039/C5RA01851J, U.S. Pat. No. 8,660,669 (Feb. 25 2014), CA 2685161 A. (Oct. 18, 2007), and US 20120156648 (Jun. 21, 2012), the contents of each of which are incorporated herein by this reference.

In the depicted embodiment, there are typically six (6) points of contact on top and bottom with 3 on each side with the gums for contacting the top and bottom jaws simultaneously. This number can vary, and embodiments having, for example, four top and bottom contact points are included. The contact points are optimally positioned in line with the centers of resistance (“COR”) of the teeth. Each contact point may be at a different height and corresponds to the COR of the teeth in its location. The center of resistance position for the delivery of bioelectric signals is for the most efficient tooth movement. Signaling from the COR allows for the entire are of the alveolus to be stimulated with the ideal signal strength.

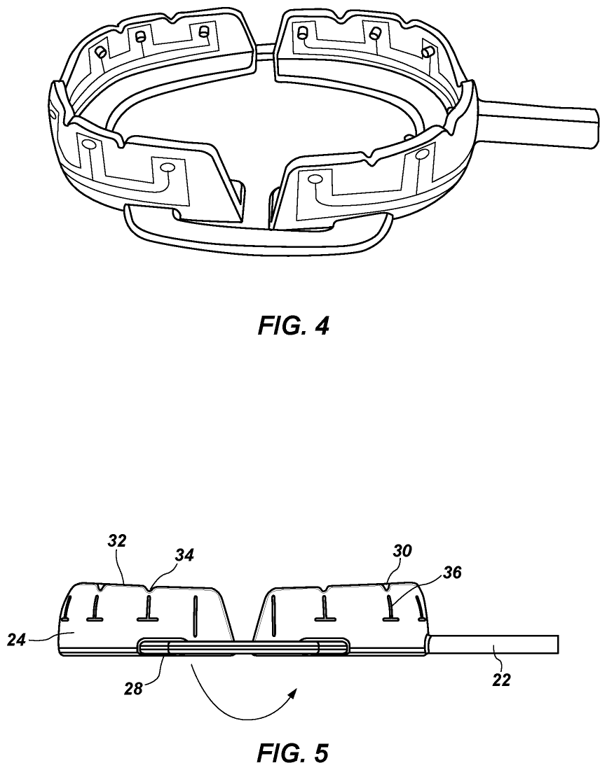

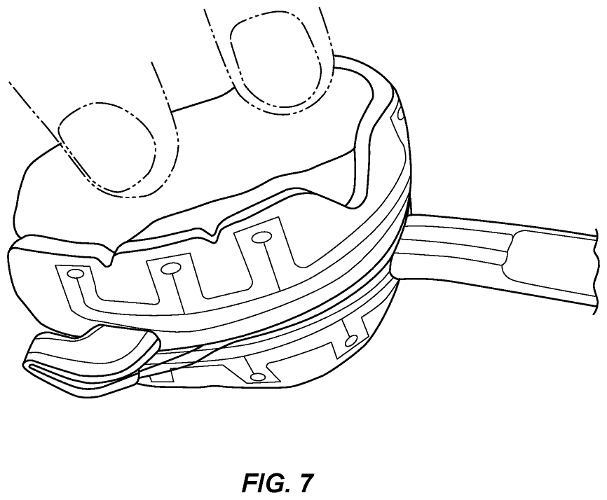

As shown in FIG. 3, the mouthpiece has bendable hinge portions 28, which also electrically connect the portions of the mouthpiece proximal 30 and distal 32 the bioelectric stimulator for delivery of the bioelectric signal(s). In use, the mouthpiece may be first folded back upon itself (see FIGS. 5 and 7) before placement into the subject's mouth.

In the depicted mouthpieces, the hinges allow for positive pressure to keep the mouthpiece in place without having to bite down fully. Also in the depicted mouthpieces, there is a “V” cut out 42 in the front of the mouthpiece on top and bottom for the superior and inferior frenulums (FIG. 2).

In certain embodiments, the hinge portions 28 may be cut completely through (e.g., in the case where only the top or bottom teeth are being subjected to the orthodontic procedure.)

The mouthpiece of FIG. 4 is shown with the circuitry (e.g., electrically conductive leads and “wiring” and leads contained within the mouthpiece 24) for delivering the bioelectric signal(s) from the bioelectric stimulator to contact pins 26 for application of the selected bioelectric signal(s) to the subject's gums.

FIG. 5 shows a dental mouthpiece in an open, unfolded orientation, where the two portions 30, 32 lay flat upon a plane. The dental mouthpiece folds at the center of the hinge portion 28 back upon itself (see FIG. 7).

For help fitting the mouthpiece 24 to the subject's mouth there are notches (or “vertical depressions”) 34 placed above corresponding pre-cut portions 36. During placement of the mouthpiece into the subject's mouth, the, for example, orthodontist can cut, e.g., with a scissor (not shown) from the notch to the corresponding pre-cut portion 36, which thus opens up the pre-cut portion (not shown) allowing the mouthpiece to be customized for the patient's teeth.

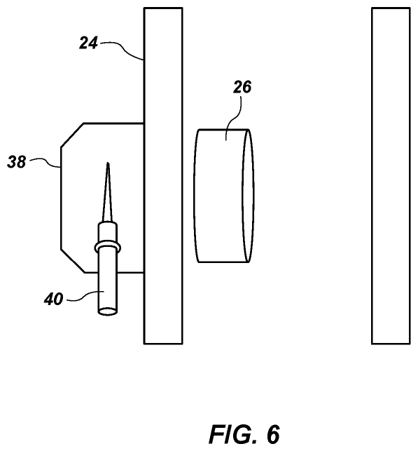

FIG. 6 is a close up view of a contact pin 26 placed within a connector housing 38 and speared by a lead in a dental mouthpiece 24 as described herein. The connector housing 38 is sized and shaped to accommodate the contact pin 26. The depicted contact pin 26 can be inserted in an aperture passing through the mouthpiece 24 and to the connector housing 38. Preferably, the contact pin 26 can be replaced and/or adjusted or moved for depth. The aperture is preferably of approximately the same size as the contact pin 26 to accommodate it snugly. The lead is in electrical communication via the mouthpiece 24 (e.g., via wiring or other circuitry as shown in FIGS. 4, 6, and 7) with the bioelectric stimulator.

FIG. 7 shows the dental mouthpiece of FIG. 4 folded back upon itself. For example, immediately before insertion into the subject's mouth (not shown).

Once fitted into the patient's mouth, bioelectric stimulation is used to improve the medical procedure. During application of the bioelectric signals, conductive sponges and gels may be used to improved conduction contact. Adding a teaspoon of salt helps conduction properties.

The bioelectric signals are generally selected to cause the subject's tissues to, for example, upregulate expression and/or release of a protein selected from the group consisting of SDF-1, M-CSF, RANKL, OPG, VEGF, IGF-1, TNF-α, eNOS and any combination thereof.

RANKL binds to the RANK receptor on the mesenchymal precursor cells to differentiate into osteoclasts which are responsible for bone resorption/demineralization. TNF-α is another pathway similar to RANKL, and acts in much the same way to cause differentiation of osteoclastic precursors into osteoclasts. VEGF increases blood supply by forming additional blood vessels to initially carry away the minerals and mineral salts during the resorption process (demineralization), on the leading side of tooth movement, and then carry the minerals back to the areas for remineralization on the trailing side, during tooth movement. Bringing these sequences of cells and proteins together can reduce up to 300%, the amount of time needed to wear orthodontic braces to finish the teeth straightening procedure.

It has been shown that RANKL injections accelerated by ⅔rds tooth movement and OPG—Osteoprotegerin—injections served to freeze tooth positions after movement. Zupan et al. “The relationship between osteoclastogenic and anti-osteoclastogenic pro-inflammatory cytokines differs in human osteoporotic and osteoarthritic bone tissues,” Journal of Biomedical Science, 2012, 19:28 (DOI: 10.1186/1423-0127-19-28), the contents of which are incorporated herein by this reference. However, two to three time weekly needle injections would need to be done by a doctor in an orthodontist's office are not well tolerated by most patients, have risk of infection, cause pain, and have a high cost.

The described device and method produces the same volume of RANKL protein and OPG as the needle injection studies with only two 20 minute bioelectric protein expression sessions a week. The stimulation is pain free in fact it reduces any pain from tooth movement that may be present. The stimulation can be done in the subject's home, e.g., while watching TV or reading conveniently at a relatively low cost. There is virtually no risk of infection.

The device described herein provides sustainable optimal upregulated expression and/or release with an increase in the quantity of the right cells and proteins over time and in the right sequence to optimize tooth movement by accelerating bone resorption/demineralization at the leading edge of the tooth during movement. This acceleration phenomenon is responsible for being able to shorten orthodontic treatment time significantly. Also, it can produce orthodontic tooth movement acceleration, post-orthodontic tooth stabilization, and craniofacial bone graft healing acceleration and it has been shown that the teeth are able to move more rapidly, with research indicating an increased rate of up to 300%.

The bioelectric stimulator is also used to enhance bone stability following orthodontic tooth movement utilizing the bone deposition pathway. In the same manner as for bone resorption, the specific proteins stimulate the specific cells to differentiate into osteoblasts (bone deposition cells) and thereby increase the quantity and quality of bone surrounding the teeth after tooth movement. This can be done rapidly by expressing the right proteins and cells at the right time to cut the stability time by up to two thirds.

In certain embodiments, the method includes: placing a bioelectric stimulator having electrically associated therewith a mouthpiece constructed to fit covering the teeth and against the dental gums of a subject via the mouthpiece. The bioelectric stimulator is attached to and/or in electrical association with a mouthpiece that fits adjacent the respective teeth and gums of the subject.

The bioelectric stimulator and mouthpiece cause SDF-1 upregulated expression and/or release in the subject as a cell homing signal to recruit mesenchymal stem cells from bone marrow and dental gums to become osteoclastic precursor cells. The stimulator causes SDF-1 upregulated expression and/or release as a cell homing signal to recruit mesenchymal stem cells from bone marrow and gingival tissue (gums) to become osteoclastic precursor cells.

The bioelectric stimulator and mouthpiece cause M-CSF upregulated expression and/or release in the subject as a cell homing signal to recruit osteoclastic precursor cells from bone marrow and dental gums to differentiate into osteoclasts. The M-CSF is a cell homing signal to recruit osteoclastic precursor cells from bone marrow and gingival tissue (gums) to differentiate into osteoclasts.

Typically, one set of signals from the bioelectric stimulator will attract the cells in SDF-1 and M-CSF to increase the numbers of osteoclastic progenitor cells to the area of tooth movement.

The bioelectric stimulator and mouthpiece cause an increase in the level of RANKL in the subject to allow the osteoclastic precursor cells to become osteoclasts and increase the rate of bone resorption/demineralization. The increased expression level of RANKL allows the osteoclastic precursor cells to become osteoclasts and multinucleated osteoclasts and thereby increase the bone resorption process.

The bioelectric stimulator and mouthpiece cause VEGF to increase blood vessels and blood supply in the subject to carry the necessary proteins and minerals and mineral salts needed for bone resorption/demineralization and osteosynthesis. VEGF increases blood vessel formation and blood supply to carry the necessary proteins and mineral salts needed for bone resorption (softening).

The bioelectric stimulator and mouthpiece cause IGF-1 upregulated expression and/or release in the subject, which increases the rate of bone metabolism for bone resorption/demineralization and then the re-mineralization process.

The bioelectric stimulator and mouthpiece cause upregulated expression and/or release of TNF-α in the subject to help osteoclast differentiation, function and survival for the process of resorption/demineralization of bone on the leading edge of tooth movement.

The bioelectric stimulator and mouthpiece cause OPG upregulated expression and/or release to enhance osteoblast formation and bone formation/re-mineralization for tooth stability following orthodontic tooth movement.

Relationship Between The Components:

The bioelectric stimulator sends specific signal(s) to the tissue for cell and protein expression typically via the mouthpiece.

SDF-1 and M-CSF recruit an increased expression of osteoclastic progenitor cells to the area of tooth movement.

Another set of signals cause the over expression of TNF-α and RANKL, which directs the pre-osteoclastic cells to differentiate into additional osteoclasts and thereby accelerates the resorption (softening) of bone due to the increase in the number of progenitor cells with activating proteins. Historically, the rate of tooth movement is limited by the amount of RANKL present and number of preosteoclasts available to permit osteoclast formation and cause bone resorption, at the leading (compression) side of tooth movement.

VEGF promotes angiogenesis, increasing the ability of the tissue to remove minerals and mineral salts during the resorption/demineralization process. VEGF speeds up the process of bone metabolism for the resorption/demineralization and then bone formation re-mineralization.

The release of eNOS nitric oxide synthase improves local blood flow. eNOS and VEGF are responsible for carrying the mineral salts away during the resorption process, which allows the bone to be demineralized (softened) and the tooth to move through the bone. The more the bone resorbs, the more blood vessels are needed to carry the mineral salts away, to allow for a substantial increase in tooth movement.

OPG causes the bone to re-mineralize and the teeth to stabilize in their orthodontically corrected positions. The stimulator and mouthpiece cause an increased release of OPG following the completion of tooth movement, to enhance tooth/bone stability by stimulating increased osteoblastic activity with additional tooth stabilizing bone deposition. This signal is utilized following orthodontic tooth movement to enhance tooth stability through an increase of bone deposition (hardening). The signal will stimulate an increase of osteoblastic activity (greater number of progenitor cells and increased expression of OPG) to strengthen the bone following the completion of tooth movement. This will substantially increase the rate of bone deposition which will lead to improved tooth/bone stability in a significantly shorter period of time.

By using the stimulator to increase the number of osteoclastic cells and specific proteins and by combining these effects in a sequential way, the rate of bone resorption/demineralization is increased. This will result in accelerating tooth movement and therefore a decrease in the length of time for orthodontic treatment.

The device and method calls for signaling (in sequence) for recruiting stem cells, promoting differentiation into osteoclasts through the release of specific proteins, and enhancing the growth of additional blood vessels to achieve the acceleration of bone resorption/demineralization for the shortening of orthodontic treatment time. A further micro pump (not shown) is optional and may be used for severe craniofacial anomaly cases.

By stimulating the release of the protein OPG (RANKL antagonist), the osteoclastic bone resorption process is halted, and the progenitor cells then become osteoblasts that are responsible for bone remineralization. This facilitates orthodontic stability after the tooth movement portion of treatment is completed.

A bioelectric stimulator is associated with a mouthpiece placed in the mouth for a minimum of 20-40 minutes a day up to 3 days a week. The mouthpiece portion can conduct electricity by, e.g., being made of a conductive polymer, having a conductive hydrogel included, by using a conductive tape or wrap, and/or by using conductive metal elements (e.g., contact pins) built into the mouthpiece in strategic positions.

The bioelectric stimulator is programmed to cause the cells of a subject an altered expression of to release a permutation of one or more of the following proteins: SDF-1, M-CSF, RANKL, TNF-α, VEGF, HGF, IGF-1, eNOS, Klotho, TGF-B1, OPG, etc. in sequence.

Additionally, the device may be used to help with facial bone graft/reconstruction for people with craniofacial anomalies (cleft lip and palates). It may also be useful in helping to heal surgeries to the mouth and skull including various titanium, titanium alloy, or ceramic type implants.

Bioelectric signals given herein may be adjusted in view of the impedance of the subject's jaw, which may be measured by an impedance analyzer or multimeter.

Generally, the system hereof involves a bioelectric stimulator that controls upregulated expression and/or release of RANKL, TNF-α, OPG, SDF-1, HGF, IGF-1, VEGF, eNOS, Klotho, TGF-B1, and M-CSF. SDF-1 is generally for recruiting stem cells and maturing blood vessels. IGF-1 is for DNA repair. VEGF grows blood vessels. eNOS dilates blood vessels.

What follows are preferred signals, which may be applied in any permutation during a series of 20-40 minute treatment cycles up to 3 times a week until desired tooth movement is complete.

VEGF—angiogenesis uses a 50 Hz biphasic signal with a pulse width duration that falls within the range of 200 μs to 300 μs. The amplitude may be adjusted to a comfortable level based on the patient's somatosensory response for a continuous signal delivery of no less than 1 minute (preferably 5 minutes).

SDF-1—Stem cell recruiting signal uses a 30 Hz biphasic signal with a pulse width duration that falls within the range of 50 μs to 150 μs. The amplitude may be adjusted to a comfortable level based on the patient's somatosensory response for a continuous signal delivery of no less than 1 minute (preferably 5 minutes).

Stem cell proliferation signals use a 1 to 2 Hz (approximately 70 pulses per minute) biphasic signal of low amplitude (500 pA to 500 μA) for up to 3 hours followed by a 0.33-0.5 Hz signal (20 pulses per minute) with a high amplitude pulse signal (e.g., 1-6 volts), and a pulse width duration within the range of 0.2-0.7 milliseconds for up to 3 hours.

IGF-1—promote bone and normal tissue growth uses a uses a 22 Hz positive monophasic signal with a pulse width duration that falls within a 10% to 50% duty cycle. The amplitude may be adjusted to a comfortable level based on the patient's somatosensory response, but typically remains in a range of less than 1 mA for a continuous signal delivery of no less than 1 minute (preferably 5 minutes).

RANKL/TNF Receptor activator of nuclear factor kappa-B (NF-κB) ligand/TNF-α promotes osteoclastogenisis and subsequent bone degradation uses a 2/100 Hz frequency modulated biphasic signal with either an oscillating duration of approximately 7 seconds or a carrier/envelope frequency relationship between the two signals. The signal is delivered with a 1 ms +/−0.5 ms pulse width duration. The amplitude may be adjusted to a comfortable level based on the patient's somatosensory response for a continuous signal delivery of no less than 1 minute (preferably 10 to 15 minutes). Optional use depending on application to be followed by 15 Hz, 1 Gauss EM field, consisting of 5-millisecond bursts with 5-microsecond pulses followed by 200-μs pulse duration at 30 Hz and with current amplitude of 140 mA. This would typically be conducted in an orthodontic office setting.

A bioelectric signal that produces osteoprotegerin (or “OPG”; also known as osteoclastogenesis inhibitory factor (OCIF), or tumor necrosis factor receptor superfamily member 11B (TNFRSF11B), is a protein that in humans is encoded by the TNFRSF11B gene) by a bioelectric signal range of 3 mV to 5 mV at frequency range 1 to 3 MHz duration range 30 to 40 mW/cm² for a minimum of 20 to 45 minutes.

eNOS—improves vascular tone and uses alternating high-frequency (HF) and medium-frequency (MF) signals that comprise symmetric, biphasic, trapezoid pulses, with 400-μs pulse duration. HF consisted of 75 Hz pulses for 6 seconds on, 21 seconds off with a 1.5/1-second ramp-up/ramp-down duration, respectively, for a minimum of 1 minute (preferably 15 minutes). MF consisted of 45 Hz pulses with 5 seconds on, 12 seconds off with similar ramp-up/ramp-down durations for a minimum of 1 minute (preferably 15 minutes). An alternative, or follow-on, signal may include a 1 Hz biphasic stimulation applied for 9 seconds, followed by a 1 second silent period for 20 minutes and/or a 20 Hz biphasic stimulation applied for 2 seconds, followed by silent period for 28 seconds for 20 min.

Klotho promotes a myriad of beneficial regenerative processes, including site-specific stabilization of osteoclast number and surface area, thereby promoting bone resorption. The klotho signal uses a 20 Hz biphasic pulse with a pulse width duration in the range of 1 ms to 7.8 ms. The amplitude may be adjusted to a comfortable level based on the patient's somatosensory response, but typically remains in a range of less than 0.1 mV to 1 V for a continuous signal delivery of no less than 1 minute (preferably 5 minutes or greater). This bioelectric signal also upregulates the expression of RANKL.

In certain embodiments, the bioelectric stimulator is programmed to produce either a single signal or a sequential train of electrical signals comprising any permutation of the signals proposed herein to be applied at various patient-tolerable amplitudes and for any duration.

In certain embodiments, bioelectric signals for RANKL and VEGF are applied for accelerated tooth movement, and then the bioelectric signal for OPG is applied, with a separate device for stabilization. If the treatment cycle is 20 minutes, RANKL is preferably applied for about 15 minutes and VEGF for about 5 minutes. In certain embodiments, there is a 44 to 46% increase in RANKL in the treated teeth and gums.

In certain embodiments, the OPG bioelectric signal is preferably applied for 20 minutes straight in multiple sessions for stabilization after the teeth have been straightened (“post-treatment stabilization”). The upregulation of OPG promotes bone formation and stabilizes teeth positions straight with minimal to no use of retainers.

In certain embodiments, the patient undergoes an application of 7 to 35 minutes, preferably 20 minutes, stimulation twice a week, resulting in teeth straightening much faster than without the therapy, and with far less pain and discomfort. In certain cases, instead of 18 to 36 months of therapy, the patient's teeth have been straightened in 3 to 6 months. In certain cases, 60% of the treated patients had straight teeth in three months.

In certain cases, reports of pain and discomfort were reduced by 70%. In certain cases, after the teeth have been straightened, they are kept so without the need for retainers.

The invention is further described with the aid of the following illustrative Example(s).

### EXAMPLES

### Example I

Orthodontic braces and clear aligners work by applying force to teeth in order to gradually realign them. This force causes a demineralization (softening) of the bone, which allows the tooth to move. Although the time it takes for patients to wear braces or aligners varies considerably, it generally takes on average about 2 years. The described system (see, e.g., FIG. 1) however utilizes bioelectric energy to significantly increase the rate at which teeth move. The system is a removable and non-invasive appliance that a patient wears in his or her mouth for 20-40 minutes every 3 to 4 days. Alternatively, the described bioelectric stimulation mouthpiece may be worn only 3 times a week for 20 minutes per session.

The bioelectric stimulator emits small electric pulses that control (e.g., upregulate) expression of RANKL, SDF-1, HGF, IGF-1, TNF-α and VEGF, eNOS and M-CSF and OPG as well as stem cell differentiation. Studies have been completed for all these cells and proteins individually for various applications of regeneration. Previously, studies demonstrated that regular needle injections of RANKL in the area of desired tooth movement, significantly accelerated tooth movement and therefore decreases the time needed to wear braces or aligners.

This bioelectric stimulator achieves much quicker orthodontic treatment results with less pain. Also, the electrical stimulation reduces pain in itself. When compared to the well-documented tooth movement acceleration approach of using surgical corticotomies, the described system and method is faster, while removing any morbidity along with eliminating the pain and suffering of surgery. The key to the increased rate is drawing an abundance of the needed cells and proteins to the site of tooth movement to accelerate the demineralization (softening) and re-mineralization (hardening) of bone, thereby allowing teeth to move faster.

One example is SDF-1, a key signal for homing stem cells from the surrounding tissue (bone marrow, gum tissue, fat cells and circulating blood) to come to the treated site to aide in tooth movement. There are many other cells and proteins and cytokines that have an increased expression through specific patented signals, all working to substantially increase the rate of tooth movement.

The described system addresses the desire to reduce (e.g., by half) the time it takes to treat orthodontic patients. The approach is to speed up the normal process of bone demineralization in order to accelerate tooth movement. This described system is completely different than previous devices as it provides clear cut direct control for the release of essential cells and proteins needed for accelerating tooth movement, and with less pain.

The bioelectric stimulator can be programmed to lead to over-expression of SDF-1, which recruits critical progenitor cells and proliferates them in the area of orthodontic tooth movement forces. Concurrently, the progenitor cells are acted upon by the proteins over-expressed via the stimulator. As the increasing number of osteoclastic progenitor cells and the increasing specific proteins combine, the net effect is an increase in the number of osteoclasts. These cells are responsible for the demineralization of the bone and are known to be the limiting factor in tooth movement. The greater number of osteoclasts, the greater the resorption/demineralization and the greater the rate of tooth movement.

As the bone is demineralized, there is a need to remove the mineral salts away from the area. This is achieved by signaling for enhance growth of blood vessels and improved blood flow. This increase in blood vessel growth allows the minerals that are a byproduct of bone resorption to be carried away from the site of bone resorption. As the demineralization reaches a critical amount, tooth movement will take place. The increase rate of bone resorption results in an acceleration of tooth movement and therefore a decrease in the length of time needed for orthodontic treatment.

Once the tooth movement is finalized, the same bioelectric stimulator is programmed to increase the amount of bone remineralization. The pathway is to have an increase of the progenitor cells signaled to the area. Specific proteins can be over expressed simultaneously to act on the progenitor cells to cause the differentiation into osteoblasts, which are responsible for bone deposition. As with the bone resorption pathway, the greater the number of osteoblastic cells the greater the bone deposition. This can result in accelerating the tooth/bone stability and therefore decrease the length of time need for retention. The greater the stability, the less chance for relapse.

The bioelectric stimulator accurately delivers a multitude of signals to the gums and bone. The stimulator is programmed with the correct signals in the proper sequence to facilitate initially bone resorption (softening) followed by bone deposition (remineralization).

The two-pronged approach first works to accelerate tooth movement. The device and method are used for proper signaling with the proper sequence for recruiting stem cells, having them differentiate into osteoclasts, by the release of certain proteins, and to grow additional blood vessels are all necessary for the acceleration of bone resorption and shortening orthodontic treatment time.

The second part of the approach creates greater tooth stability following active tooth movement. The device and method is then used for a different signaling, with the proper sequence for recruiting stem cells, have them differentiate into osteoblasts by the release of certain proteins, and to grow additional blood vessels are all necessary for the acceleration of bone deposition and the shortening of the orthodontic retention time.

The only interchangeable parts are the bioelectric stimulator and the mouthpiece which is a conductive polymer. Different stimulators can be used as well as various types of materials for the mouthpiece to deliver the signals.

Once orthodontic treatment commences, a bioelectric stimulator is attached to a conductive polymer mouthpiece and is placed in the mouth every third day for 20-40 minutes. The mouthpiece is designed to cover one or both of the dental arches. In certain embodiments, the stimulator is programmed to cause upregulated expression and/or release of SDF-1, MCSF, RANKL, TNF-α, VEGF, and eNOS, in sequence, during the active portion of treatment. Once the active orthodontic treatment is completed, the same mouth piece is re-programmed to cause upregulated expression and/or release of SDF-1, OPG, VEGF, eNOS to trigger cell differentiation for the remineralization process and enhanced accelerated tooth stability.

### Example II

Over a period of three months, a prospective, randomized, double blind, sham controlled, two-arm study (44 patients enrolled, 29 patients (n=29) completed the study) inducing alignment was conducted. The Test Group had 60% of the patients (who had been diagnosed with more severe crowding than the Control Group) have perfectly straight teeth vs. of 14% of patients in the Control Group. The Control Group had braces and mouthpiece. The Test Group had braces, mouthpiece, and bioelectric stimulation. Treatment was for 20 minutes, twice a week. Tooth movement was more translation with the Test Group and, in the control group, more tipping. Tooth movement was assessed after three months of treatment.

The Test Group had more crowding and greater space gain at months 2 and 3. (p=0.01 at 2 mos. and p=0.006 at 3 mos.) The Test Group had significantly less pain at 24, 48, and 72 hours (up to 70% less) following orthodontic adjustments.

With the use of bioelectric stimulation 3 to 4 weeks of treatment was equivalent to 6 months of conventional treatment, with complete alignments after 6 months and a 70% decrease in treatment discomfort.

### REFERENCES

(The contents of the entirety of each of which is incorporated herein by this reference.)

Almpani and Kantarci “Nonsurgical Methods for the Acceleration of the Orthodontic Tooth Movement,” Tooth Movement. Front Oral Biol., vol. 18, pp 80-91 (Karger, Basel, CH 2016) (DOI: 10.1159/000382048), Published online: Nov. 24, 2015.

Atkinson et al. “Bioelectric Properties of the Tooth” 1969 Volume: 48 issue: 5, page(s): 789-794.

Chang et al. “Pulsed electromagnetic fields stimulation affects osteoclast formation by modulation of osteoprotegerin, RANK ligand and macrophage colony-stimulating factor,” Journal of Orthopaedic Research, 23 (2005) 1308-1314.

d'Apuzzo et al. “Biomarkers of Periodontal Tissue Remodeling during Orthodontic Tooth Movement in Mice and Men: Overview and Clinical Relevance,” The Scientific World Journal, Vol. 2013 (2013), Article ID 105873, 8 pages, http://dx.doi.org/10-155/2013/105873.

Dibart et al. “Tissue response during Piezocision-assisted tooth movement: a histological study in rats,” Eur. J Orthod. (2014) 36 (4): 457-464; DOI: https://doi.org/10.1093/ejo/cjt079.

El-Bialy et al. “Effect of Low Intensity Pulsed Ultrasound (LIPUS) on Tooth Movement and Root Resorption: A Prospective Multi-Center Randomized Controlled Trial” J. Clin. Med. 2020, 9, 804; doi:10.3390/jcm9030804.

Fonseca et al. “Electrical stimulation: Complementary therapy to improve the performance of grafts in bone defects?” Journal of Biomedical Materials Research Part B: Applied Biomaterials 2018 Vol 000b, Issue 0.

Giganti et al. “Changes in serum levels of TNF-α, IL-6, OPG, RANKL and their correlation with radiographic and clinical assessment in fragility fractures and high energy fractures,” J. Biol. Regul. Homeost. Agents, 2012 Oct-Dec; 26(4): 671-80.

Gurbax S, Raahat V S, Roopsirat K, Devinder P S. Accelerated Orthodontic Tooth Movement: A Review. Mod Res Dent. 1(2). MRD.000508. 2017.

R. Hamman “Modulation Of RANKL and Osteoprotegerin in Adolescents Using Orthodontic Forces,” Masters Thesis, University of Tennessee (2010).

K. Hart, Katherine Ann D. D. S., “RANKL and Osteoprotegerin Levels in Response to Orthodontic Forces” (2012), Theses and Dissertations (ETD). Paper 107. http://dx.doi.org/10.21007/etd/cghs.2012.0127.

Holding et al. “The correlation of RANK, RANKL and TNF-α expression with bone loss volume and polyethylene wear debris around hip implants,” Biomaterials 27(30): 5212-9⋅November 2006.

Hudson et al. “Local delivery of recombinant osteoprotegerin enhances postorthodontic tooth stability” Calcif Tissue Int. 2012 Apr; 90(4): 330-42. doi: 10.1007/s00223-012-9579-4.

Iglesias-Linares et al. “The use of gene therapy vs. corticotomy surgery in accelerating orthodontic tooth movement.” Orthod Craniofac Res. 2011 Aug; 14(3): 138-48. doi: 10.1111/j.1601-6343.2011.01519.x.

Jansen et al. “Stimulation of osteogenic differentiation in human osteoprogenitor cells by pulsed electromagnetic fields: an in vitro study” BMC Musculoskeletal Disorders (2010) 11: 188 DOI: 10.1186/1471-2474-11-188.

Kanzaki et al. “Local OPG gene transfer to periodontal tissue inhibits orthodontic tooth movement.” J. Dent. Res. 2004; 83:920-925.

Kanzaki et al. “Periodontal ligament cells under mechanical stress induce osteoclastogenesis by receptor activator of nuclear factor kappaB ligand up-regulation via prostaglandin E2 synthesis.” J. Bone Miner. Res. 2002; 17:10-220.

Kanzaki et al. “Local RANKL gene transfer to the periodontal tissue accelerates orthodontic tooth movement,” Gene Therapy (2006) 13, 678-685.

Kaur et al. “Electrically conductive polymers and composites for biomedical applications,” RSC Adv., 2015, 5, 37553-37567 DOI: 10.1039/C5RA01851J.

Kim, D., Park, Y., Kang, S. The effects of electrical current from a micro-electrical device on tooth movement. The Korean Journal of Orthodontics. 2008 Oct 30: 38(5); 337-346.

Vinod Krishnan, Ze'ev Davidovitch (eds.), Biological Mechanisms of Tooth Movement, (John Wiley & Sons 2015).

Keles et al. “Inhibition of tooth movement by osteoprotegerin vs. pamidronate under conditions of constant orthodontic force,” Eur. J. Oral Sci. 2007 Apr; 115(2): 131-6.

Khan et al. “Accelerating Tooth Movement: What Options We Have?” J. Dent. Health Oral Disord. Ther. 2016, 5(7): 00181.

Kondo et al. “Types of tooth movement, bodily or tipping, do not affect the displacement of the tooth's center of resistance but do affect the alveolar bone resorption” Angle Orthod 2017 Jul; 87(4): 563-569.

Li, C., Chung, C .J., Hwang, C., et al. Local injection of RANKL facilitates tooth movement and alveolar bone remodelling. Oral Diseases. 2019; 25: 550-560.

Nimeri et al. “Acceleration of tooth movement during orthodontic treatment—a frontier in Orthodontics,” Prog. Orthod. 2013; 14:42; DOI: 10.1186/2196-1042-14-42.

Norton et al. “Bioelectric Perturbations of Bone: Research Directions and Clinical Applications” Angle Orthod (1984) 54 (1): 73-87.

Otero et al. “Expression and Presence of OPG and RANKL mRNA and Protein in Human Periodontal Ligament with Orthodontic Force,” Gene-Regulation-and-Systems-Biology, 2016, 10, 15-20.

Seifi & Jeszri “Correlation of bone resorption induced by orthodontic tooth movement and expression of RANKL in rats,” Dental Journal, Vol. 26, No. 4 (2009).

Showkatbakhsh et al. “The effect of pulsed electromagnetic fields on the acceleration of tooth movement.” World J Orthod. 2010 Winter; 11(4): e52-6.

Showkatbakhsh, A., Younessian, F., Dianat, O., et al. Effect of Intra-Canal Direct Current Electric Stimulation on Orthodontic Tooth Movement: An Experimental Study in Canines. Journal of Dental School 2016; 34(3): 157-67.

Signature Orthodontics “Accelerated Tooth Movement,” http:/sigortho.com/accelerated-tooth-movement.

Spadari, G. S., Zaniboni, E., Vedovello, S. A. S. et al. “Electrical stimulation enhances tissue reorganization during orthodontic tooth movement in rats” Clin Oral Invest (2017) 21: 111.

Tan et al. “Bioelectric Perturbations in Orthodontic tooth movement” 2010 Journal of Dental Sciences & Research 1: 1: Pages 41-49.

Tokyo Medical and Dental University “RANKL expressed by osteocytes has an important role in orthodontic tooth movement” Science Daily Oct. 20, 2017,

Verna et al. “The rate and the type of orthodontic tooth movement is influenced by bone turnover in a rat model” European Journal of Orthodontics 22 (2000) 343-352.

Walsh & Choi “Biology of the RANKL-RANK-OPG System in Immunity, Bone, and Beyond,” Front Immunol. 2014; 5: 51.

M. Yamaguchi, “RANK/RANKL/OPG during orthodontic tooth movement,” Orthod. Craniofac. Res. 2009 May; 12(2): 113-9. doi: 10.1111/j.1601-6343.2009.01444.x.

Chia-Ping Yang “Effect RANKL Produced by Periodontal Ligament Cells on Orthodontic Tooth Movement” (2016) Dental Theses. Paper 13.

Zaniboni et al. “Do electrical current and laser therapies improve bone remodeling during an orthodontic treatment with corticotomy?” Clin Oral Invest 23, 4083-4097 (2019). https://doi.org/10.1007/s00784-019-02845-9.

Zhao et al. “Local osteoprotegerin gene transfer inhibits relapse of orthodontic tooth movement.” Am J Orthod Dentofacial Orthop. 2012 Jan; 141(1):30-40. doi: 10.1016/j.ajodo.2011.06.035.

Zupan et al. “The relationship between osteoclastogenic and anti-osteoclastogenic pro-inflammatory cytokines differs in human osteoporotic and osteoarthritic bone tissues,” Journal of Biomedical Science, 2012, 19:28 (DOI: 10.1186/1423-0127-19-28).

What is claimed is:

1. A device useful in an orthodontic procedure of a subject, the device comprising:
a bioelectric stimulator programmed to produce one or more bioelectric signals that are delivered by an oral apparatus comprising:
a mouthpiece comprising a polymer surface-contacting material and constructed to fit over the subject's teeth, braces, and/or aligners, and
conductive electrode nodules positioned within the mouthpiece in proximity of the subject's gums,
wherein the mouthpiece further comprises circuitry able to deliver a bioelectric signal or signals to the conductive electrode nodules, wherein a first bioelectric signal thereof is:
a 2/100 Hz frequency modulated biphasic signal with either an oscillation duration of approximately 7 seconds or a carrier/envelope frequency relationship between the two signals, wherein the signal is delivered with a 1 ms +/−0.5 ms pulse width duration.

2. The device of claim 1, wherein the amplitude may be adjusted to a comfortable level based on the subject's somatosensory response for a continuous signal delivery of no less than 1 minute.

3. The device of claim 1, wherein the bioelectric stimulator is further programmed to produce a subsequent biphasic bioelectric signal of 20 Hz with a pulse width duration in the range of 1 ms to 7.8 ms, and wherein the amplitude thereof remains in a range of less than 0.1 mV to 1 V for a continuous signal delivery of no less than 1 minute,

4. The device of claim 1, wherein the bioelectric stimulator is further programmed to produce a subsequent biphasic bioelectric signal of 30 Hz with a pulse width duration that falls within the range of 50 μs to 150 μs for a continuous signal delivery of greater than 1 minute.

5. The device of claim 1, wherein the bioelectric stimulator is further programmed to produce a subsequent biphasic bioelectric signal of 50 Hz with a pulse width duration that falls within the range of 200 μs to 300 μs for a continuous signal delivery of no less than 1 minute.

6. The device of claim 1, wherein the bioelectric stimulator is further programmed to produce a subsequent bioelectric signal that uses alternating high-frequency (HF) and medium-frequency (MF) signals that comprise symmetric, biphasic, trapezoid pulses, with 400-μs pulse duration. HF consisted of 75 Hz pulses for 6 seconds on, 21 seconds off with a 1.5/1-second ramp-up/ramp-down duration, respectively, for a minimum of 1 minute, wherein the MF comprises 45 Hz pulses with 5 seconds on, 12 seconds off, with ramp-up/ramp-down durations for a minimum of 1 minute.

7. The device of claim 1, wherein the bioelectric stimulator is further programmed to produce a subsequent bioelectric signal of 15 Hz, 1 Gauss EM field, consisting of 5-millisecond bursts with 5-microsecond pulses followed by 200 μs pulse duration at 30 Hz.

8. The device of claim 1, wherein the bioelectric stimulator is further programmed to produce a subsequent bioelectric signal of 40 Hz, with a pulse width duration of 100 μs.

9. The device of claim 1, wherein the bioelectric stimulator is further programmed to produce a subsequent positive monophasic bioelectric signal of 22 Hz with a pulse width duration that falls within a 10% to 50% duty cycle. The amplitude may be adjusted to a comfortable level based on the patient's somatosensory response, but typically remains in a range of less than 1 mA for a continuous signal delivery of no less than 1 minute.

10. The device of claim 1, wherein the bioelectric stimulator is further programmed to produce a subsequent bioelectric or ultrasonic signal at frequency range 1 MHz to 3 MHz with a power density within the range of 30 to 40 mW/cm².

11. A method of assisting in an orthodontic procedure in a subject, the method comprising:
placing the device of claim 1 over the subject's teeth (with associated braces and aligners) in proximity of the gums of the subject via a mouthpiece and applying electrical stimulation to the gums as part of an orthodontic procedure.

12. A method of assisting in an orthodontic procedure in a subject of the type involving applying braces or aligners to the subject's teeth, the method comprising:
obtaining a device comprising:
a bioelectric stimulator programmed to produce sequential electrical signals, wherein a first electrical signal of said sequential electrical signals is a biphasic pulse of 0.1 Volt at 20 Hz and a 7.8 ms pulse duration, and,
electrically associated with the bioelectric stimulator, an electrically conductive mouthpiece comprised of a polymer and constructed to fit over the subject's teeth and in proximity of the subject's gums,
placing the device over the subject's teeth, and applied braces or aligner(s), and in proximity of the dental gums of the subject via the electrically conductive mouthpiece, and
applying the first electrical signal to the dental gums of the subject as part of the orthodontic procedure.

13. The method according to claims 12, further comprising utilizing the device to produce a subsequent electrical signal that upregulates expression of stem cell homing factor (“SDF-1”) in the subject.

14. The method according to claims 12, further comprising utilizing the device to produce a subsequent electrical signal that upregulates expression of vascular endothelial growth factor (“VEGF”) in the subject.

15. The method according to claims 12, further comprising utilizing the device to produce a subsequent electrical signal that upregulates expression of insulin-like growth factor (“IGF-1”) in the subject.

16. The method according to claims 12, further comprising utilizing the device to produce a subsequent electrical signal that upregulates expression of osteoprotegerin (“OPG”) in the subject.

17. The method according to claims 12, further comprising utilizing the device to produce a subsequent electrical signal that upregulates expression of eNOS in the subject.

18. The method according to claims 12, wherein the orthodontic procedure comprises applying braces to the subject's teeth.

19. The method according to claims 12, wherein the orthodontic procedure comprises applying an aligner to the subject's teeth.

20. A mouthpiece comprising first and second portions that fold upon one another via a flexible hinge or hinges, wherein, when folded, the mouthpiece is sized to fit within a subject's mouth, the mouthpiece having circuitry that extends from an integrated or external bioelectric stimulator to a contact point or contact points placed so as to interact with the subject's gums and deliver a bioelectric signal thereto.
